(19) United States
(12) Patent Application Publication
(10) Pub. No.: US 2004/0048278 A1
(43) Pub. Date: Mar. 11, 2004

(54) Diagnosis of diseases associated with the human c-mos gene

(76) Inventors: Alexander Olek, Berlin (DE); Christian Piepenbrock, Berlin (DE); Kurt Berlin, Stahnsdorf (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/416,111

(22) PCT Filed: Nov. 6, 2001
(86) PCT No.: PCT/EP01/12831

(30) Foreign Application Priority Data
Nov. 6, 2000 (DE) 100 54 972.1

(51) Int. Cl. C12Q 1/68; C07H 21/04; C12P 19/34

(52) U.S. Cl. 435/6; 536/23.1; 435/91.2

(57) ABSTRACT

The invention relates to the chemically modified genomic sequence of the human C-mos gene, to oligonucleotides and/or PNA oligomers directed against the sequence for detecting the cytosine methylation condition of the human C-mos gene and to a method for determining genetic and/or epigenetic parameters of the human C-mos gene.

### DESCRIPTION

1. Field of the Invention

The levels of observation that have been well studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers and to a method for the diagnosis and/or therapy of diseases which have a connection with the genetic and/or epigenetic parameters of the oncogene humos and, in particular, with the methylation status thereof.

2. Prior Art

The Moloney murine sarcoma virus (MSV) belongs to the replication-inactive retroviruses that transform fibroblasts in culture, and in vivo induce sarcomas. The virus was generated by the recombination between the Moloney murine leukaemia virus and a sequence derived from mouse-cells. The segment of the MSV stemming from mouse-cells, also designated as v-mos, is required for the induction and maintenance of the viral transformation. Homologous genes to v-mos are c-mos that likewise stems from the mouse, and the human c-mos (humos), that, due to the evolutionary conservation of viral oncogenes among the vertebrate species, could be mapped on the human chromosome 8q11-12 (Prakash K, McBride O W, Swan D C, Devare S G, Tronick S R, Aaronson S A. Molecular cloning and chromosomal mapping of a human locus related to the transforming gene of Moloney murine sarcoma virus. Proc Natl Acad Sci USA. 1982 September; 79(17): 5210-4; Watson R, Oskarsson M, Vande Woude G F. Human DNA sequence homologous to the transforming gene (mos) of Moloney murine sarcoma virus. Proc Natl Acad Sci USA. 1982 July; 79(13):4078-82.). The c-mos proto-oncogene is expressed in a significant numbers of lung carcinomas, and probably plays a role in their development (Athanasiou A, Gorgoulis V G, Zacharatos P, Mariatos G, Kotsinas A, Liloglou T, Karameris A, Foukas P, Manolis E N, Field J K, Kittas C. c-mos immunoreactivity is an indicator of good prognosis in lung cancer. Histopathology. 2000 July; 37(1):45-54). An association with laryngeal cancer is likewise assumed (Dolcetti R, Pelucchi S, Maestro R, Rizzo S, Pastore A, Boiocchi M. Proto-oncogene allelic variations in human laryngeal cell carcinomas of the larynx. Eur Arch Otorhinolaryngol. 1991;248(5):279-85). In contrast, the participation of c-mos in acute myeloblastic leukaemia is controversly discussed (Morris C M, Bowen J, Fitzgerald P H. Localization of human c-mos to chromosome band 8q11 in leukemic cells with the t(8;21) (q22;q22). Hum Genet. 1989 March; 81(4):339-42). Furthermore, the participation of c-mos in chronic myelocystic leukaemie (Huerre C, Despoisse S, Gilgenkrantz S, Lenoir G M, Junien C. c-Ha-rasl is not deleted in aniridia-Wilms' tumour association. Nature. Oct. 13-19, 1983; 305(5935):638-41) as well as in Burkitt's lymphoma is assumed (Klein G. The role of gene dosage and genetic transpositions in carcinogenesis. Nature. Nov. 26, 1981; 294(5839):313-8).

-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. Dec. 15, 1996;24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. Jun. 15, 1997;25(12):2529-31, WO Application 95/00669) or by enzymatic digestion (Xiong Z, Laird P W. COBRA: a sensitive and quantitative DNA methylation assay. Nucleic Acids Res. Jun. 15, 1997;25(12):2532-4). In addition, detection by hybridisation has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Charlton J, Bird AP, Walter J, Reik W. Methylation analysis on individual chromosomes: improved protocol for bisulphite genomic sequencing. Nucleic Acids Res. Feb. 25, 1994;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. May 19, 1995;157(1-2):261-4; WO 97 46705, WO 95 15373 and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption lonisation Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. Oct. 15, 1988;60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption lonisation Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. Apr. 25, 1995;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### OBJECT OF THE INVENTION

The present invention shall provide oligonucleotides and/or PNA-oligomers for the detection of cytosine-methylations, and provide a method that is particularly suited for the diagnosis of genetic and epigenetic parameters of the gene humos. The present invention is based on the finding that, in particular, the cytosine methylation patterns are suitable for the diagnosis of diseases associated with humos.

### DESCRIPTION

It is therefore an object of the present invention to provide the chemically modified DNA of the gene humos, as well as to provide oligonucleotides and/or PNA-oligomers for the detection of cytosine-methylations, as well as to provide a method that is particularly suited for the diagnosis of genetic and epigenetic parameters of the gene humos. The invention is based on the finding that genetic and epigenetic parameters, and, in particular, the cytosine methylation pattern of the gene humos is particularly suited for the diagnosis of diseases associated with humos.

This objective is achieved according to the present invention by a nucleic acid, comprising a sequence of at least 18 bases in length of the chemically pretreated DNA of the gene humos according to one of Seq. ID No. 1 to Seq. ID No.4. The chemically modified nucleic acid could heretofore not be connected with the ascertainment of genetic and epigenetic parameters.

The object of the present invention is further achieved by an oligonucleotide or oligomer for detecting the cytosine methylation state in chemically pretreated DNA, containing at least one base sequence having a length of at least 13 nucleotides which hybridises to a chemically pretreated DNA of the gene humos according to one of Seq. ID No. 1 to Seq. ID No.4. The oligomer probes according to the present invention constitute important and effective tools which, for the first time, make it possible to ascertain the genetic and epigenetic parameters of the gene humos. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which comprise at least one oligomer for each of the CpG dinucleotides of one of the sequences of Seq. ID No. 1 to Seq. ID No.4. Preferred is a set which comprises at least one oligomer for each of the CpG dinucleotides from one of the Seq ID No. 1 to Seq ID No.4.

Moreover, the present invention makes available a set of at least two oligonucleotides which can be used as so-called “primer oligonucleotides” for amplifying DNA sequences of one of Seq. ID No. 1 to Seq. ID No. 4, or segments thereof.

In the case of the sets of oligonucleotides according to the present invention, it is preferred that at least one oligonucleotide is bound to a solid phase.

The present invention moreover relates to a set of at least 10 n (oligonucleotides and/or PNA-oligomers) used for detecting the cytosine methylation state in chemically pretreated genomic DNA (Seq. ID No.1 through Seq. ID No.4). These probes enable diagnosis and/or therapy of genetic and epigenetic parameters of the gene humos. The set of oligomers may also be used for detecting single nucleotide polymorphisms (SNPs) in the chemically pretreated DNA of the gene humos according to one of Seq. ID No.1 through Seq. ID No.4.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with diseases associated with humos in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the analysis of diseases associated with humos which comprises at least one nucleic acid according to the present invention. DNA chips are known, for example, from U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to an 18 base long segment of the base sequences specified in the appendix (Seq. ID No. 1 to Seq. ID No.4), oligonucleotides and/or PNA-oligomers as well as instructions for carrying out and evaluating the described method. However, a kit along the lines of the present invention can also contain only part of the aforementioned components.

The present invention also makes available a method for ascertaining genetic and/or epigenetic parameters of the gene humos by analysing cytosine methylations and single nucleotide polymorphisms, including the following steps:

In the first step of the method, a genomic DNA sample is chemically treated in such a manner that cytosine bases which are unmethylated at the 5′-position are converted to uracil, thymine, or another base which is dissimilar to cytosine in terms of hybridisation behaviour. This will be understood as ‘chemical pretreatment’ hereinafter.

The genomic DNA to be analysed is preferably obtained form usual sources of DNA such as cells or cell components, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, or combinations thereof.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

Fragments of the chemically pretreated DNA are amplified, using sets of primer oligonucleotides according to the present invention, and a, preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two oligonucleotides whose sequences are each reverse complementary or identical to an at least 18 base-pair long segment of the base sequences specified in the appendix (Seq. ID No. 1 to Seq. ID No.4). The primer oligonucleotides are preferably characterised in that they do not contain any CpG dinucleotides.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualised by means of matrix assisted laser desorption/ionisation mass spectrometry (MALDI) or using electron spray mass spectrometry (ESI).

The amplificates obtained in the second step of the method are subsequently hybridised to an array or a set of oligonucleotides and/or PNA probes. In this context, the hybridisation takes place in the manner described in the following. The set of probes used during the hybridisation is preferably composed of at least 10 oligonucleotides or PNA-oligomers. In the process, the amplificates serve as probes which hybridise to oligonucleotides previously bonded to a solid phase. The non-hybridised fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5^{th} to 9^{th} nucleotide from the 5′-end of the 13-mer. One oligonucleotide exists for each CpG dinucleotide. Said PNA-oligomers contain at least one base sequence having a length of 9 nucleotides which is reverse complementary or identical to a segment of the base sequences specified in the appendix, the segment containing at least one CpG dinucleotide. The cytosine of the CpG dinucleotide is the 4^{th} to 6^{th} nucleotide seen from the 5′-end of the 9-mer. One oligonucleotide exists for each CpG dinucleotide.

In the fourth step of the method, the non-hybridised amplificates are removed.

In the final step of the method, the hybridised amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualised by means of matrix assisted laser desorption/ionisation mass spectrometry (MALDI) or using electron spray mass spectrometry (ESI).

The produced fragments may have a single positive or negative net charge for better detectability in the mass spectrometer. The aforementioned method is preferably used for ascertaining genetic and/or epigenetic parameters of the gene humos.

The oligomers according to the present invention or arrays thereof as well as a kit according to the present invention are intended to be used for the diagnosis of a disease associated with humos by analysing methylation patterns of the gene humos. According to the present invention, the method is preferably used for the diagnosis of important genetic and/or epigenetic parameters within the gene humos.

The method according to the present invention is used, for example, for the diagnosis of cancerous diseases, such as lung carcinoma, laryngeal cancer, acute myoblastic leukaemia, chronical myelotic leukaemia or Burkitt's lymphoma.

In addition, the nucleic acids according to the present invention of Seq. ID No.1 to Seq. ID No.4 can be used for the diagnosis of genetic and/or epigenetic parameters of the gene humos.

The present invention moreover relates to a method for manufacturing a diagnostic agent for the diagnosis of diseases associated with humos by analysing methylation patterns of the gene humos, the diagnostic agent and/or therapeutic agent being characterised in that at least one nucleic acid according to the present invention is used for manufacturing it, possibly together with suitable additives and auxiliary agents.

A further subject matter of the present invention relates to a diagnostic agent for diseases associated with humos by analysing methylation patterns of the gene humos, comprising at least one nucleic acid according to the present invention, possibly together with suitable additives and auxiliary agents.

The present invention moreover relates to the diagnosis and/or prognosis of events which are disadvantageous to patients or individuals in which important genetic and/or epigenetic parameters within the gene humos wherein said parameters obtained by means of the present invention may be compared to another set of genetic and/or epigenetic parameters, the differences serving as the basis for a diagnosis and/or prognosis of events which are disadvantageous to patients or individuals.

In the context of the present invention the term “hybridisation” is to be understood as a bond of an oligonucleotide to a completely complementary sequence along the lines of the Watson-Crick base pairings in the sample DNA, forming a duplex structure. To be understood by “stringent hybridisation conditions” are those conditions in which a hybridisation is carried out at 60° C. in 2.5×SSC buffer, followed by several washing steps at 37° C. in a low buffer concentration, and remains stable.

The term “functional variants” denotes all DNA sequences which are complementary to a DNA sequence, and which hybridise to the reference sequence under stringent conditions and have an activity similar to the corresponding polypeptide according to the present invention.

In the context of the present invention, “genetic parameters” are mutations and polymorphisms of the gene humos and sequences further required for their regulation. To be designated as mutations are, in particular, insertions, deletions, point mutations, inversions and polymorphisms and, particularly preferred, SNPs (single nucleotide polymorphisms). Nevertheless, polymorphisms can also be insertions, deletions or inversions.

In the context of the present invention, “epigenetic parameters” are, in particular, cytosine methylations and further chemical modifications of DNA bases of the gene humos and sequences further required for their regulation. Further epigenetic parameters include, for example, the acetylation of histones which, however, cannot be directly analysed using the described method but which, in turn, correlates with the DNA methylation.

In the following, the present invention will be explained in greater detail on the basis of the sequences and examples without being limited thereto.

Seq. ID No. 1 shows the sequence of the chemically pretreated genomic DNA of the gene humos

Seq. ID No.2 shows the sequence of a second chemically pretreated genomic DNA of the gene humos

Seq. ID No.3 shows the reverse complementary sequence of Seq. ID No. 1 of the chemically pretreated genomic DNA of the gene humos

Seq. ID No.4 shows the reverse complementary sequence of Seq. ID No. 2 of the chemically pretreated genomic DNA of the gene humos

Seq. ID No.5 shows the sequence of an oligonucleotide for amplifying humos from example 1

Seq. ID No.6 shows the sequence of a second oligonucleotide for amplifying humos from example 1

Seq. ID No.7 shows the sequence of an oligonucleotide for hybridising the amplificate of humos from example 1

Seq. ID No.8 shows the sequence of a second oligonucleotide for hybridising the amplificate of humos from example 1

Seq. ID No.9 shows the sequence of a third oligonucleotide for hybridising the amplificate of humos from example 1

Seq. ID No.10 shows the sequence of a fourth oligonucleotide for hybridising the amplificate of humos from example 1

Seq. ID No. 1 shows the sequence of an oligonucleotide for hybridising the amplificate of humos from example 1

Seq. ID No.12 shows the sequence of a fifth oligonucleotide for hybridising the amplificate of humos from example 1

Seq. ID No.13 shows the sequence of an oligonucleotide for hybridising the amplificate of humos from example 1

Seq. ID No.14 shows the sequence of a sixth oligonucleotide for hybridising the amplificate of humos from example 1

Seq. ID No.15 shows the sequence of a seventh oligonucleotide for hybridising the amplificate of humos from example 1

Seq. ID No.16 shows the sequence of an eighth oligonucleotide for hybridising the amplificate of humos from example 1

The following example relates to a fragment of the gene humos, in which a specific CG-position is to be analysed for its methylation status.

### EXAMPLE 1

### Performing the Methylation Analysis in the Gene Humos

### Example 1

### Performing the Methylation Analysis in the Gene Humos

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behaviour while the cytosines methylated at the 5-position remain unchanged.

If bisulfite solution is used in a concentration range between 0.1 and 6 M, a then an addition takes place at the non-methylated cytosine bases. Moreover, a denaturating reagent or solvent as well as a radical interceptor is present. A subsequent alkaline hydrolysis then gives rise to the conversion of non-methylated cytosine nucleobases to uracil. This chemically converted DNA is then used for the detection of methylated cytosines. In the second method step, the treated DNA sample is diluted with water or an aqueous solution. Preferably, the DNA is subsequently desulfonated (10-30 min, 90-100° C.) at an alkaline pH value. In the third step of the method, the DNA sample is amplified in a polymerase chain reaction, preferably using a heat-resistant DNA polymerase.

In the present case, cytosines of the gene humos, in this case from the promoter region, and Exon 1, respectively, are examined. Using sequences of this gene, the cell lines MHH-Call2, MHH-Call4, BV-173, 380, NALM-6, and REH (all of the type human B cell precursor leukaemia) of CDl9+B-cells, and CCRF-CEM, Jurkat, Molt-17, P12-Ichikawa (all human T cell leukaemia), RPMI-8402 (human T cell acute lymphoblastic leukaemia), and Karpas-299 (human T cell lymphoma), respectively, can be distinguished from CD4+CD8-T-cells. To this end, a defined fragment having a length of 494 bp is amplified with the specific primer oligonucleotides TTTATTGATTGGGAGTAGGT (Seq. ID No. 5) and CTAATTTTACAAACATCCTA (Seq. ID No. 6). This amplificate serves as a sample which hybridises to an oligonucleotide previously bonded to a solid phase, forming a duplex structure, for example CCTTACTACGTTAAACTC (Seq. ID No. 7) or CCTTACTACATTAAACTC (Seq. ID No. 8), the cytosine to be detected being located at position 164 of the amplificate. The methylated cytosine is determined with the oligonucleotide (Seq. ID No. 7), which has a guanine at the respective complementary position, whereas the unmethylated form that is represented by a thymine is determined with the oligonucleotide (Seq. ID No. 8), which has an adenine at the respective complementary position. Further oligonucleotides that can be used for hybridisation, contain the following sequences: GTTACCACCGAACTCCAT (Seq. ID No. 9) and GTTACCACCAAACTCCAT (Seq. ID No. 10) with the cytosine to be determined at position 263 of the amplificate, CTCCCCTACGTCCCCCTC (Seq. ID No. 11) and CTCCCCTACATCCCCCTC (Seq. ID No. 12) with the cytosine to be determined at position 323 of the amplificate, CTCCAATACGACAATAAA (Seq. ID No. 13) and CTCCAATACAACAATAAA (Seq. ID No. 14) with the cytosine to be determined at position 89 of the amplificate, AAACAAACCGTTCACAAC (Seq. ID No. 15) and AAACAAACCATTCACAAC (Seq. ID No. 16) with the cytosine to be determined at position 401 of the amplificate.

The detection of the hybridisation product is based on Cy5 flourescently labelled primer oligonucleotides which have been used for the amplification. The hybridisation reaction of the amplified DNA with the oligonucleotide takes place only if a methylated cytosine was present at this location in the bisulfite treated DNA. Thus, the methylation status of the specific cytosine to be analysed may be inferred from the hybridisation product.

### EXAMPLE 2

### DIAGNOSIS OF DISEASES ASSOCIATED WITH HUMOS

In order to relate the methylation patterns to one of the diseases associated with humos, for example, laryngeal cancer, acute myelotic leukaemia, chronic myelotic leukaemia and Burkitt's lymphoma, it is initially required to analyse the DNA methylation patterns of a group of diseased and of a group of healthy patients. These analyses are carried out, for example, analogously to example 1. The results obtained in this manner are stored in a database and the CpG dinucleotides which are methylated differently between the two groups are identified. This can be carried out by determining individual CpG methylation rates as can be done, for example, in a relatively imprecise manner, by sequencing or else, in a very precise manner, by a methylation-sensitive “primer extension reaction”. It is also possible for the entire methylation status to be analysed simultaneously, and for the patterns to be compared, for example, by clustering analyses which can be carried out, for example, by a computer.

Subsequently, it is possible to allocate the examined patients to a specific therapy group and to treat these patients selectively with an individualised therapy.

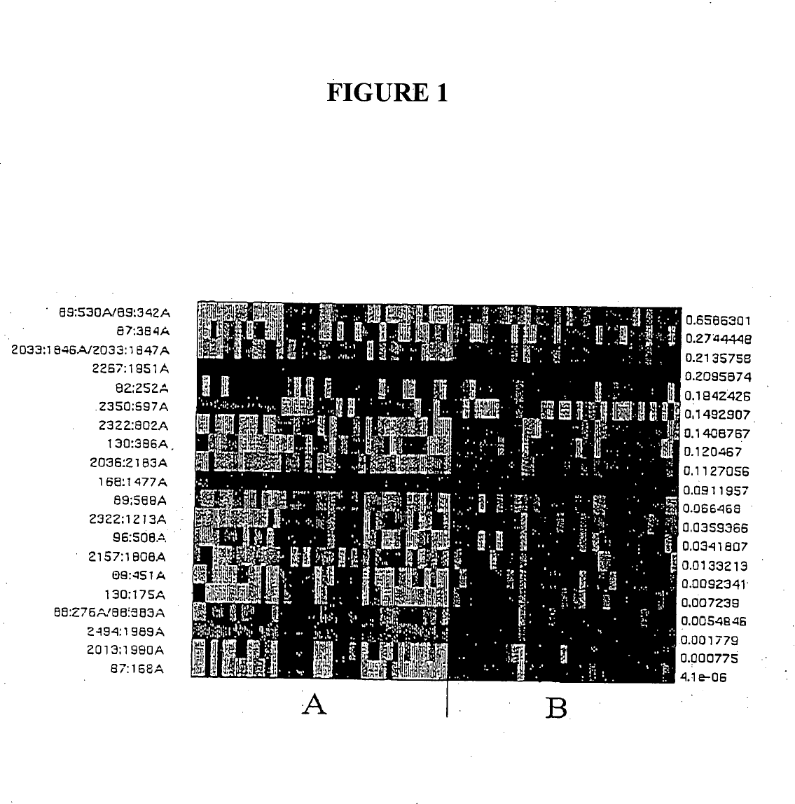

The invention shall furthermore be explained with respect to the following examples and sequences and examples, wherein FIG. 1 shows the differentiation of mantle cell lymphoma (MCL), diffuse large B-cell lymphoma (DLBCL) and chronic lymphatic leukaemia (CLL/SLL) with follicular lymphoma I (FL I well differentiated) and follicular lymphoma II (FL II medium differentiated). A high probability for methylation corresponds to red signals (darker in the Figure) a lower probability green signals (lighter in the Figure) and black intermediate values. The samples on the left side of the FIG. 1(A) were assigned to the group of MCL, DLBCL, and CLL/SLL, the ones on the right side (B) FL I, and II (see also example 4).

Seq. ID No. 17 shows the sequence of an oligonucleotide as used in example 3.

Seq. ID No. 18 shows the sequence of an oligonucleotide as used in example 3.

Seq. ID No. 19 shows the sequence of an oligonucleotide as used in example 3.

Seq. ID No. 20 shows the sequence of an oligonucleotide as used in example 3.

### EXAMPLE 3

### Methylation Analysis Within the Gene Humos

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behaviour while the cytosines methylated at the 5-position remain unchanged. If bisulfite solution is used, then an addition takes place at the non-methylated cytosine bases. Moreover, a denaturating reagent or solvent as well as a radical interceptor is present. A subsequent alkaline hydrolysis then gives rise to the conversion of non-methylated cytosine nucleobases to uracil. This chemically converted DNA is then used for the detection of methylated cytosines. In the second step of the method, the treated DNA sample is diluted with water or an aqueous solution. Preferably, the DNA is subsequently desulfonated. In the third step of the method, the DNA sample is amplified in a polymerase chain reaction, preferably using a heat-resistant DNA polymerase. Die PCR reactions were performed in a thermocycler (Eppendorf GmbH). For a 25 μl sample, 10 ng DNA, 0.08 μM of each primer oligonucleotide, 1,6 mM dNTPs and one Unit HotstartTaq were used. The other conditions were chosen according to the instructions of the manufacturer. For the PCR, first a denaturation for 15 minutes at 96° C., thereafter 46 cycles (60 seconds at 96° C., 45 seconds at 52° C., and 75 seconds at 72° C.) and a final elongation of 10 minutes at 72° C. were performed. The presence of the PCR-products was checked on agarose gels.

In the present case, cytosines of the gene humos are examined. Using sequences of this gene, samples of patients with the diagnosis mantle cell lymphoma (MCL), diffuse large B-cell lymphoma (DLBCL), and chronic lymphatic leukaemia (CLL/SLL) can be distinguished from patients with the diagnosis follicular lymphoma I (FL I well differentiated) and follicular lymphoma II (FL II slightly differentiated). For this, a defined fragment having a length of 523 bp is amplified using the specific primer oligonucleotides TGATTGGGAGTAGGTGTGTT (Seq. ID No. 17) and CAAATCTTCCAACTTCTCAAA (Seq. ID No. 18 This amplificate serves as a probe that hybridises to an oligonucleotide previously bound to a solid phase by forming a duplex-structure, for example TATGGAGTTCGGTGGTAA (Seq. ID No. 19) or TATGGAGTTTGGTGGTAA (Seq. ID No. 20), wherein the cytosine to be determined is present at position 259 of the amplificate. The methylated cytosine is determined with the oligonucleotide (Seq. ID No. 19) that has a guanine at the respective complementary position, whereas the umnethylated form that is represented by a thymine, is determined with the oligonucleotide (Seq. ID No. 20) that has an adenine at the respective complementary position. The detection of the hybridisation product is based on Cy5 flourescently labelled primer oligonucleotides which have been used for the amplification. The hybridisation reaction of the amplified DNA with the oligonucleotide takes place only if a methylated cytosine was present at this location in the bisulfite treated DNA. Thus, the methylation status of the specific cytosine to be analysed may be inferred from the hybridisation product.

### EXAMPLE 4

### Digital Phenotype

The following example describes the comparison of mantle cell lymphoma (MCL), diffuse large B-cell lymphoma (DLBCL) and chronic lymphatic leukaemia (CLL/SLL) with follicular lymphoma I (FL I well differentiated) and follicular lymphoma II (FL II slightly differentiated). For the multiplex PCRs, fluorescently labelled primers were used in order to amplify 8 fragments per reaction. All PCR products of each individual were mixed and hybridised to glass object slides, that carried a pair of immobilised oligonucleotides at each position. Each of these detection oligonucleotides were designed in order to hybridise it against bisulfite-converted sequences present at CpG-sites, which were present in the either initial unmethylated (TG) or methylated (CG) status. The hybridisation conditions were chosen for the detection of differences at single nucleotides of the variants TG and CG. The ratios of both signals were calculated based on the comparison of the intensities of the fluorescent signals.

The information is subsequently detected in a ranked matrix (cf. FIG. 1) in relation to the CpG methylation differences between two classes of tissues. The most significant cp.-positions are depicted at the lower end of the matrix, with the significance decreasing in the direction of the upper end. Dark grey (in the original Figure: red) indicates a high degree of methylation, light grey (in the original Figure: green) a low one, and black an intermediate degree of methylation. Each row represents a specific CpG-position in one gene and each column shows the methylation profile of different CpGs for one sample. On the left side, a gene identifier is given; the corresponding name of the respective gene can be found in table 1. The corresponding accession numbers of the genes are listed in table 1. The number in front of the colon indicates the gene name and the number behind the colon the specific oligonucleotide. On the right side of FIG. 1, the p-values of the individual CpG-positions are shown. The p-values represent the probabilities whether the observed allocation occurs randomly or not.

The first (in FIG. 1 on the left) group contained 42 samples of both gender versus 38 samples of the second (in FIG. 1 present on the right side) group. The p-value weighted methylation indicates a clear differentiation between the two groups, 9 CpG positions (red and grey shades of colour, respectively) of 7 different genes were significantly distinguished (corrected p-value <0.05) between the two groups. The cross-validated accuracy of the classification, determined by SVM (support vector machine) (F. Model, P. Adorjan, A. Olek, C. Piepenbrock, Feature selection for DNA methylation based cancer classification. Bioinformatics. 2001 June; 17 Suppl 1: S 157-64), is calculated as 78,8% with a standard deviation of 2,8%. In the context of a larger study, here the gene humos was examined, which is represented by the gene identifier 89.

TABLE 1

| Gene Number | Gene Name | Accession Number |
|---|---|---|
| 82 | EGR4 | NM_001965 |
| 87 | AR | NM_000044 |
| 88 | CDK4 | NM_000075 |
| 89 | HUMOS | NM_005372 |
| 96 | RB1 | NM_000321 |
| 130 | GPIbbeta | NM_000407 |
| 168 | MYOD1 | NM_002478 |
| 2013 | BCL2 | NM_000633 |
| 2033 | CDKN1A | NM_000389 |
| 2036 | CDKN2B | NM_004936 |
| 2157 | MLH1 | NM_004936 |
| 2267 | TGFBR2 | NM_003242 |
| 2322 | TP73 | NM_005427 |
| 2350 | CDKN1C | NM_000076 |
| 2494 | BAK1 | NM_001188 |

1. A nucleic acid comprising a sequence at least 18 bases in length of a segment of the chemically pretreated DNA of the gene humos according to one of the Seq. ID No. 1 to Seq. ID No. 4.

2. Oligomer (oligonucleotide or peptide nucleic acid (PNA)-oligomer) for the detection of the cytosine methylation status in chemically pretreated, comprising in each case at least one base sequence having a length of at least 9 nucleotides which hybridises to a chemically pretreated DNA of the gene humos according to one of the Seq. ID No 1 to Seq. ID No 4.

3. The oligomer as recited in claim 2; wherein the base sequence comprises at least one CpG dinucleotide.

4. The oligomer as recited in claim 3; characterised in that the cytosine of the CpG dinucleotide is located approximately in the middle third of the oligomer.

5. A set of oligomers as recited in claim 3, comprising at least one oligomer for the detection of the cytosine methylation status of at least one of the CpG dinucleotides from one of the sequences of the Seq. ID No. 1 to Seq. ID No. 4.

6. A set of oligomers as recited in claim 3, comprising at least one oligomer for the detection of the cytosine methylation status of all CpG dinucleotides from one of the sequences of the Seq. ID No. 1 to Seq. ID No. 4.

7. A set of at least two oligonucleotides as recited in claim 2 which can be used as primer oligonucleotides for the amplification of DNA sequences of one of Seq. ID No. 1 to Seq. no. 4, or segments thereof.

8. A set of oligonucleotides as recited in claim 7, characterised in that at least one oligonucleotide is bound to a solid phase.

9. A set of oligomers for the detection of the cytosine methylation status and/or of single nucleotide polymorphisms (SNPs) in a chemically pretreated genomic DNA according to one of the sequences Seq. ID No. 1 to Seq. ID No. 4, comprising at least ten of the oligomers according to claims 2 to 4.

10. A method for manufacturing an arrangement of different oligomers (array) fixed to a carrier material for analysing diseases associated with the methylation state of the CpG dinucleotides of one of the Seq. ID No. 1 to Seq. ID No. 4, wherein at least one oligomer according to any of claims 2 to 4 is coupled to a solid phase.

11. An arrangement of different oligomers (array) according to one of claims 2 to 4.

12. An array of different oligonucleotide- and/or PNA-oligomer sequences as recited in claim 11, characterised in that these are arranged on a plane solid phase in the form of a rectangular or hexagonal lattice.

13. The array as recited in any of claims 11 or 12, characterised in that the solid phase surface is composed of silicon, glass, polystyrene, aluminium, steel, iron, copper, nickel, silver, or gold.

14. A DNA- and/or PNA-array for analysing diseases associated with the methylation state of genes, comprising at least one nucleic acid according to one of the preceding claims.

15. A method for ascertaining genetic and/or epigenetic parameters for the diagnosis of existing diseases or the predisposition to specific diseases by analysing cytosine methylations, characterised in that the following steps are carried out:
a) in a genomic DNA sample, cytosine bases which are unmethylated at the 5-position are converted, by chemical treatment, to uracil or another base which is dissimilar to cytosine in terms of hybridisation behaviour;
b) fragments of the chemically pretreated genomic DNA are amplified using sets of primer oligonucleotides according to claim 7 or 8 and a polymerase, the amplificates carrying a detectable label;
c) Amplificates are hybridised to a set of oligonucleotides and/or PNA probes according to the claims 2 to 4, or else to an array according to one of the claims 11 to 13;
d) the hybridised amplificates are subsequently detected.

16. The method as recited in claim 15, characterised in that the chemical treatment is carried out by means of a solution of a bisulfite, hydrogen sulfite or disulfite.

17. The method as recited in one of the claims 15 or 16, characterised in that more than ten different fragments having a length of 100-2000 base pairs are amplified.

18. The method as recited in one of the claims 15 to 17, characterised in that the amplification of several DNA segments is carried out in one reaction vessel.

19. The method as recited in one of the claims 15 to 18, characterised in that the polymerase is a heat-resistant DNA polymerase.

20. The method as recited in claim 19, characterised in that the amplification is carried out by means of the polymerase chain reaction (PCR).

21. The method as recited in one of the claims 15 to 20, characterised in that the labels of the amplificates are fluorescence labels.

22. The method as recited in one of the claims 15 to 20, characterised in that the labels of the amplificates are radionuclides.

23. The method as recited in one of the claims 15 to 20, characterised in that the labels of the amplificates are detachable molecule fragments having a typical mass which are detected in a mass spectrometer.

24. The method as recited in one of the claims 15 to 20, characterised in that the amplificates or fragments of the amplificates are detected in the mass spectrometer.

25. The method as recited in one of the claims 23 and/or 24, characterised in that the produced fragments have a single positive or negative net charge for better detectability in the mass spectrometer.

26. The method as recited in one of the claims 23 to 25, characterised in that detection is carried out and visualised by means of matrix assisted laser desorption/ionisation mass spectrometry (MALDI) or using electron spray mass spectrometry (ESI).

27. The method as recited in one of the claims 15 to 26, characterised in that the genomic DNA is obtained from cells or cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

28. A kit comprising a bisulfite (=disulfite, hydrogen sulfite) reagent as well as oligonucleotides and/or PNA-oligomers according to any of claims 2 to 4.

29. The use of a nucleic acid according to claim 1, of an oligonucleotide or PNA-oligomer according to one of the claims 2 to 4, of a kit according to claim 28, of an array according to one of the claims 10 to 13, of a set of oligonucleotides, comprising at least one oligomer for at least one of the CpG-dinucletides of one of the sequences according to Seq. ID No. 7 to Seq. ID No. 16 for the diagnosis of cancerous diseases, such as lung carcinoma, laryngeal cancer, acute myoblastic leukaemia, chronical myelotic leukaemia or Burkitt's lymphoma.

30. The use of a nucleic acid according to claim 1, of an oligonucleotide or PNA-oligomer according to one of the claims 2 to 4, of a kit according to claim 28, of an array according to one of the claims 10 to 13, of a set of oligonucleotides, comprising at least one oligomer for at least one of the CpG-dinucletides of one of the sequences according to Seq. ID No. 7 to Seq. ID No. 16 for the therapy of cancerous diseases, such as lung carcinoma, laryngeal cancer, acute myoblastic leukaemia, chronical myelotic leukaemia or Burkitt's lymphoma.

31. A kit comprising a bisulfite (=disulfite, hydrogen sulfite) reagent as well as oligonucleotides and/or PNA-oligomers according to claim 30.

32. The use of a nucleic acid according to claim 1, of an oligonucleotide or PNA-oligomer according to one of the claims 2 to 4, of a kit according to claim 28, of an array according to one of the claims 10 to 13 for the differentiation of different forms, subtypes, and phases of ALL (acute lymphatic leukaemia).


SEQUENCE LISTING (20 sequences)

SEQ ID NO: 1 - DNA, 1303 bp, Artificial Sequence
  chemically treated genomic DNA (Homo sapiens)
cggaagggaaatgtttttatttgaaagggatagttgtgttttatttcggtttttttttttatttgataaaaatttttgttgagtgatagtatagatgtagtttatttggaataagtgaaggaaaaggagaaaagggatgaggtggagcgaaggagtagttagttatgtttttaaagtttcgcggttttttttagtttttttatttattttagcggttttggtgtttttttgtaaagtgcgatgttttcgtttttggttttacgtttttattttcggagcgagttttttttatcggtggacgcgcggttttgtagtagttttttagagttatttgcgaagttgtttttgggggttattttttttcgggtttcgcggttgtcgcgtcggttggtttggtgttttattgattgggagtaggtgtgtttgttgtagaggttgggagttggagggtttggttcggtgtataaggcgatttatcgcggtgtttttgtggttataaagtaagtgaataagtgtattaagaatcgattagtatttcggcggagtttttgggttgagtttaacgtagtaaggttgcgttacgataatatcgtgcgcgtggtggttgttagtacgcgtacgttcgtagggtttaatagtttagggattattattatggagttcggtggtaacgttattttatattaagttatttatggcgtcgtcggttattttgagggggacgtaggggagttttattgtcgtattggaggatagttaagtttgggaaagtgttttaagtatttattagatgttgtgaacggtttgttttttttttattcgtaaagtattgtgtatttggatttgaagttcgcgaatattttgattagtgagtaggatgtttgtaaaattagtgatttcggttgttttgagaagttggaagatttgttgtgtttttagatatttttttattttttaggaggtatatatatttatcgcgtttcggagtttttgaaaggagagggcgtgacgtttaaagtcgatatttatttttttgttattattttttggtaaatgattattaagtaggcgtcgtattcgggggagcggtagtatatattgtacgcggtggtggtttacgatttgcgttcgttttttttcgttgtcgttttcgaggattcgtttttcgggtagcgttttggggacgttatttagcgttgttggagatttagcgcggcgtagaggtcgagcgcgcggttgtttttggtggattttatttttttgaaagttgaattcggttgattgaaaatttggttaagataag

SEQ ID NO: 2 - DNA, 1303 bp, Artificial Sequence
  chemically treated genomic DNA (Homo sapiens)
cttatcttaaccaaattttcaatcaaccgaattcaactttcaaaaaaataaaatccaccaaaaacaaccgcgcgctcgacctctacgccgcgctaaatctccaacaacgctaaataacgtccccaaaacgctacccgaaaaacgaatcctcgaaaacgacaacgaaaaaaaacgaacgcaaatcgtaaaccaccaccgcgtacaatatatactaccgctcccccgaatacgacgcctacttaataatcatttaccaaaaaataataacaaaaaaataaatatcgactttaaacgtcacgccctctcctttcaaaaactccgaaacgcgataaatatatatacctcctaaaaaataaaaaaatatctaaaaacacaacaaatcttccaacttctcaaaacaaccgaaatcactaattttacaaacatcctactcactaatcaaaatattcgcgaacttcaaatccaaatacacaatactttacgaataaaaaaaaaacaaaccgttcacaacatctaataaatacttaaaacactttcccaaacttaactatcctccaatacgacaataaaactcccctacgtccccctcaaaataaccgacgacgccataaataacttaatataaaataacgttaccaccgaactccataataataatccctaaactattaaaccctacgaacgtacgcgtactaacaaccaccacgcgcacgatattatcgtaacgcaaccttactacgttaaactcaacccaaaaactccgccgaaatactaatcgattcttaatacacttattcacttactttataaccacaaaaacaccgcgataaatcgccttatacaccgaaccaaaccctccaactcccaacctctacaacaaacacacctactcccaatcaataaaacaccaaaccaaccgacgcgacaaccgcgaaacccgaaaaaaaataacccccaaaaacaacttcgcaaataactctaaaaaactactacaaaaccgcgcgtccaccgataaaaaaaactcgctccgaaaataaaaacgtaaaaccaaaaacgaaaacatcgcactttacaaaaaaacaccaaaaccgctaaaataaataaaaaaactaaaaaaaaccgcgaaactttaaaaacataactaactactccttcgctccacctcatcccttttctccttttccttcacttattccaaataaactacatctatactatcactcaacaaaaatttttatcaaataaaaaaaaaaaccgaaataaaacacaactatccctttcaaataaaaacatttcccttccg

SEQ ID NO: 3 - DNA, 1303 bp, Artificial Sequence
  chemically treated genomic DNA (Homo sapiens)
tttattttgattaagtttttagttagtcgagtttagtttttaaagaggtgagatttattaaaagtagtcgcgcgttcggtttttgcgtcgcgttgggtttttagtagcgttggatgacgtttttaaggcgttgttcggggagcgagttttcgaagacggtagcggagagggacgggcgtaggtcgtaggttattatcgcgtatagtatgtgttgtcgttttttcgaatacggcgtttgtttggtagttatttgttagagagtgatggtaaaggaataaatgtcggttttaggcgttacgttttttttttttaggagtttcggggcgcggtgggtgtatgtgttttttagagggtaagagggtgtttggaagtatagtagattttttaattttttagagtaatcgaagttattaattttatagatattttgtttattgattaagatgttcgcgggttttaggtttaagtgtataatgttttgcgagtggaggaagagtaggtcgtttataatatttagtgagtatttgagatatttttttaaatttaattgttttttagtgcggtagtgaggtttttttgcgttttttttagggtggtcggcggcgttatagatgatttggtgtaaagtgacgttgttatcgaattttatgatgatggtttttaggttattggattttgcgggcgtgcgcgtgttggtagttattacgcgtacgatgttatcgtggcgtagttttgttacgttgagtttagtttagaaatttcgtcgagatgttagtcggtttttggtgtatttgtttatttgttttatggttataggaatatcgcggtaagtcgttttgtatatcgagttaaattttttagtttttagtttttgtagtaagtatatttgtttttagttaatggagtattaggttagtcggcgcggtagtcgcggggttcgaggaagagtggtttttagaagtagtttcgtaggtagttttgagggattgttgtagggtcgcgcgtttatcgatggggaaaattcgtttcggaggtaggggcgtagggttaggggcgagggtatcgtattttgtagggggatattagggtcgttggagtgaatgaagagattaggggaaatcgcgggattttggaaatatgattgattattttttcgttttattttatttttttttttttttttttttatttgttttaaatgagttatatttgtgttgttatttagtaagagtttttattagatggagggagaaatcggaatgaagtatagttattttttttagatgaaagtatttttttttcg

SEQ ID NO: 4 - DNA, 1303 bp, Artificial Sequence
  chemically treated genomic DNA (Homo sapiens)
cgaaaaaaaaatactttcatctaaaaaaaataactatacttcattccgatttctccctccatctaataaaaactcttactaaataacaacacaaatataactcatttaaaacaaataaaaaaaaaaaaaaaaaaaaataaaataaaacgaaaaaataatcaatcatatttccaaaatcccgcgatttcccctaatctcttcattcactccaacgaccctaatatccccctacaaaatacgataccctcgcccctaaccctacgcccctacctccgaaacgaattttccccatcgataaacgcgcgaccctacaacaatccctcaaaactacctacgaaactacttctaaaaaccactcttcctcgaaccccgcgactaccgcgccgactaacctaatactccattaactaaaaacaaatatacttactacaaaaactaaaaactaaaaaatttaactcgatatacaaaacgacttaccgcgatattcctataaccataaaacaaataaacaaatacaccaaaaaccgactaacatctcgacgaaatttctaaactaaactcaacgtaacaaaactacgccacgataacatcgtacgcgtaataactaccaacacgcgcacgcccgcaaaatccaataacctaaaaaccatcatcataaaattcgataacaacgtcactttacaccaaatcatctataacgccgccgaccaccctaaaaaaaacgcaaaaaaacctcactaccgcactaaaaaacaattaaatttaaaaaaatatctcaaatactcactaaatattataaacgacctactcttcctccactcgcaaaacattatacacttaaacctaaaacccgcgaacatcttaatcaataaacaaaatatctataaaattaataacttcgattactctaaaaaattaaaaaatctactatacttccaaacaccctcttaccctctaaaaaacacatacacccaccgcgccccgaaactcctaaaaaaaaaaaacgtaacgcctaaaaccgacatttattcctttaccatcactctctaacaaataactaccaaacaaacgccgtattcgaaaaaacgacaacacatactatacgcgataataacctacgacctacgcccgtccctctccgctaccgtcttcgaaaactcgctccccgaacaacgccttaaaaacgtcatccaacgctactaaaaacccaacgcgacgcaaaaaccgaacgcgcgactacttttaataaatctcacctctttaaaaactaaactcgactaactaaaaacttaatcaaaataaa

SEQ ID NO: 5 - DNA, 20 bp, Artificial Sequence
  chemically treated genomic DNA (Homo sapiens)
tttattgattgggagtaggt

SEQ ID NO: 6 - DNA, 20 bp, Artificial Sequence
  chemically treated genomic DNA (Homo sapiens)
ctaattttacaaacatccta

SEQ ID NO: 7 - DNA, 18 bp, Artificial Sequence
  chemically treated genomic DNA (Homo sapiens)
ccttactacgttaaactc

SEQ ID NO: 8 - DNA, 18 bp, Artificial Sequence
  chemically treated genomic DNA (Homo sapiens)
ccttactacattaaactc

SEQ ID NO: 9 - DNA, 18 bp, Artificial Sequence
  chemically treated genomic DNA (Homo sapiens)
gttaccaccgaactccat

SEQ ID NO: 10 - DNA, 18 bp, Artificial Sequence
  chemically treated genomic DNA (Homo sapiens)
gttaccaccaaactccat

SEQ ID NO: 11 - DNA, 18 bp, Artificial Sequence
  chemically treated genomic DNA (Homo sapiens)
ctcccctacgtccccctc

SEQ ID NO: 12 - DNA, 18 bp, Artificial Sequence
  chemically treated genomic DNA (Homo sapiens)
ctcccctacatccccctc

SEQ ID NO: 13 - DNA, 18 bp, Artificial Sequence
  chemically treated genomic DNA (Homo sapiens)
ctccaatacgacaataaa

SEQ ID NO: 14 - DNA, 18 bp, Artificial Sequence
  chemically treated genomic DNA (Homo sapiens)
ctccaatacaacaataaa

SEQ ID NO: 15 - DNA, 18 bp, Artificial Sequence
  chemically treated genomic DNA (Homo sapiens)
aaacaaaccgttcacaac

SEQ ID NO: 16 - DNA, 18 bp, Artificial Sequence
  chemically treated genomic DNA (Homo sapiens)
aaacaaaccattcacaac

SEQ ID NO: 17 - DNA, 20 bp, Artificial Sequence
  chemically treated genomic DNA (Homo sapiens)
tgattgggagtaggtgtgtt

SEQ ID NO: 18 - DNA, 21 bp, Artificial Sequence
  chemically treated genomic DNA (Homo sapiens)
caaatcttccaacttctcaaa

SEQ ID NO: 19 - DNA, 18 bp, Artificial Sequence
  chemically treated genomic DNA (Homo sapiens)
tatggagttcggtggtaa

SEQ ID NO: 20 - DNA, 18 bp, Artificial Sequence
  chemically treated genomic DNA (Homo sapiens)
tatggagtttggtggtaa